(19) United States
(12) Patent Application Publication
(10) Pub. No.: US 2012/0016231 A1
(43) Pub. Date: Jan. 19, 2012

(54) SYSTEM AND METHOD FOR THREE DIMENSIONAL COSMETOLOGY IMAGING WITH STRUCTURED LIGHT

(75) Inventor: ROBERT JOE WESTMORELAND, HEATH, TX (US)

(73) Assignee: MEDICAL SCAN TECHNOLOGIES, INC., HEATH, TX (US)

(21) Appl. No.: 13/184,633

(22) Filed: Jul. 18, 2011

Related U.S. Application Data
(60) Provisional application No. 61/365,339, filed on Jul. 18, 2010.

(51) Int. Cl. A61B 6/00

(52) U.S. Cl. 600/425

(57) ABSTRACT

An SLI cosmetology image sensor captures one or more images of an anatomical feature and generates a 3D surface map of the anatomical feature using SLI techniques. A feature detection module processes the 3D surface map to detect certain characteristics of the anatomical feature. Feature data of the anatomical feature is generated such as size, shape and texture. A feature analysis module processes the feature data. The feature analysis module compares the anatomical feature to prior images and feature data for the anatomical feature. The feature analysis module categorizes the anatomical feature based on templates and correlations of types of features.

### CROSS-REFERENCE TO RELATED PATENTS

The present U.S. Utility Patent Application claims priority pursuant to 35 U.S.C. §119(e) to U.S. Provisional Application Ser. No. 61/365,339, entitled “System and Method for Three Dimensional Cosmetology Imaging with Structured Light,” (Attorney Docket No. MED002), filed Jul. 18, 2010, pending, which is hereby incorporated herein by reference in its entirety and made part of the present U.S. Utility Patent Application for all purposes:

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not applicable.

### BACKGROUND OF THE INVENTION

1. Technical Field of the Invention

This invention relates to three dimensional (3D) cosmetology imaging and in particular to systems and methods for imaging using structured light illumination in the field of cosmetology.

2. Description of Related Art

Structured light illumination (SLI) techniques are a relatively low cost method for generating 3D images in biometrics, e.g. fingerprint and facial recognition. For example, one method is described in PCT Application No. WO2007/050776 entitled, “System and Method for 3D Imaging using Structured Light Illumination,” which is incorporated by reference herein. See also, U.S. Pat. No. 7,440,590 entitled, “System and Technique for Retrieving Depth Information about a Surface by Projecting a Composite Image of Modulated Light Patterns,” which is incorporated by reference herein. See also, US Published Application No. 20090103777 entitled, “Lock and Hold Structured Light Illumination,” which is also incorporated by reference herein. SLI imaging techniques have proven a cost effective solution in biometrics.

As disclosed herein, it is desirable to apply SLI techniques in other fields to provide relatively low cost and fast 3D imaging.

### BRIEF DESCRIPTION OF THE SEVERAL VIEWS OF THE DRAWINGS

FIG. 1 illustrates a schematic block diagram of an embodiment of an SLI cosmetology imaging system;

FIG. 2 illustrates a schematic block diagram of an embodiment of a Structured Light Illumination (SLI) cosmetology image sensor;

FIG. 3 illustrates a schematic block diagram of another embodiment of a Structured Light Illumination (SLI) cosmetology image sensor;

FIG. 4 illustrates a schematic block diagram of an embodiment of a projection system in a SLI cosmetology image sensor;

FIG. 5 illustrates a schematic block diagram of an embodiment of a cosmetology image camera system in a SLI cosmetology image sensor;

FIG. 6 illustrates a logical flow diagram of an embodiment of a method for capturing cosmetology images using SLI techniques;

FIG. 7 illustrates a logical flow diagram of an embodiment of a method for generating a 3D surface map from SLI cosmetology image data;

FIGS. 8A and 8B illustrate an example of a 3D surface map generated from SLI image data;

FIG. 9 illustrates a logic flow diagram of an embodiment for processing a 3D surface map to generate cosmetology data;

FIG. 10 illustrates a logic flow diagram of an embodiment for using SLI techniques in cosmetology;

FIG. 11 illustrates a logic flow diagram of an embodiment of a method for processing skin feature data captured using SLI techniques;

FIG. 12A illustrates a logic flow diagram of an embodiment for using SLI techniques in cosmetology for skin treatments; and

FIG. 12B illustrates a logic flow diagram of an embodiment of another method for processing skin feature data captured using SLI techniques.

### DETAILED DESCRIPTION OF THE INVENTION

A need exists to provide a method and system for use of Structured Light Illumination (SLI) techniques in the field of cosmetology. Cosmetology includes the study and application of treatments to enhance the appearance of hair, skin and nails. SLI cosmetology imaging systems described herein provide for cost effective and fast imaging, detection, comparison, classification and analysis of anatomical features for cosmetology. In addition, processing modules described herein provide for analysis of such SLI images for cosmetology purposes.

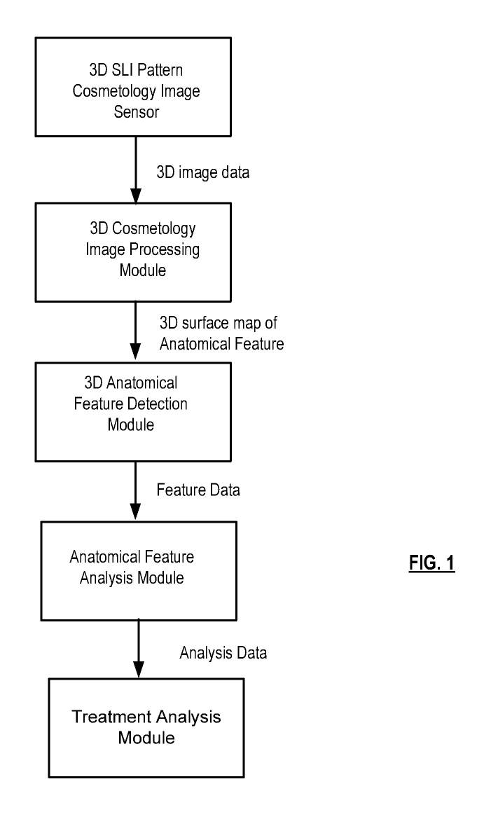

FIG. 1 illustrates a schematic block diagram of an embodiment of an SLI cosmetology imaging system. An SLI cosmetology image sensor captures one or more images of an anatomical feature and generates 3D cosmetology image data of the anatomical feature. The anatomical feature is any feature of or relating to the human body or animal body, such as hair, skin and nails or other body parts.

The 3D cosmetology image processing module processes the cosmetology image data and generates a 3D surface map of the anatomical feature. A feature detection module processes the 3D surface map to detect certain characteristics of the anatomical feature. Feature data of the anatomical feature is generated such as size, shape, color and texture. A feature analysis module processes the feature data. The feature analysis module categorizes the anatomical feature based on templates and correlations of types of features. The feature analysis module can classify the features and determine various characteristics of the features. The feature analysis module can then recommend one or more treatments or products based on the analysis. The feature analysis module may also compare the anatomical feature to prior images and feature data for the anatomical feature, for example to determine the effectiveness of a treatment or product.

The treatment analysis module processes the detected/analyzes features and determines various cosmetology treatments, including one or more products, skin treatments, hair treatments, procedures, etc.

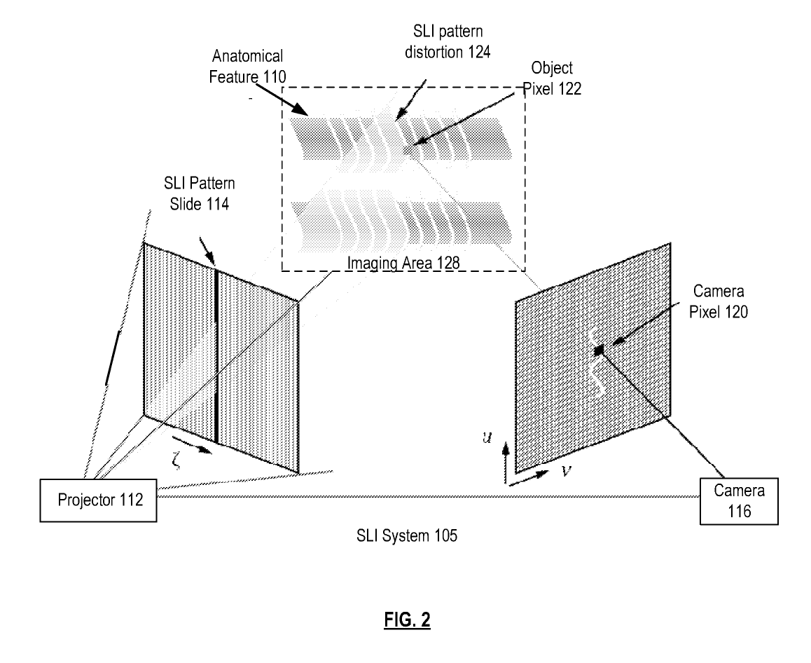

FIG. 2 illustrates a schematic block diagram of an embodiment of a Structured Light Illumination (SLI) system 105 that is implemented in the SLI cosmetology image sensor. The SLI system 105 includes an SLI pattern projector 112 and camera 116. The SLI pattern projector includes a DLP projector, LCD projector, LEDs, or other type of projector or laser. The camera 116 includes one or more digital cameras or image sensors operable to capture digital images. In operation, the image sensor/camera system is positioned and focused onto the imaging area. An SLI pattern projector projects focused light through an SLI pattern slide onto an anatomical feature 110 in imaging area 128. The SLI pattern is distorted by the surface variations of the anatomical feature as seen with SLI pattern distortion 124. While the SLI pattern is projected onto the anatomical feature 110, a camera 116 captures an image of the anatomical feature with the SLI pattern distortion 124. The camera 116 generates a frame composed of a matrix of camera pixels 120 wherein each camera pixel 120 captures image data for a corresponding object point 122 on the anatomical feature 110. The camera 116 captures one or more images of the anatomical feature 110 with the distortions in the structured light pattern. Additional SLI slide patterns may be projected onto the anatomical feature 110 while additional images are captured. The one or more 3D cosmetology images are then stored in a cosmetology image data file for processing.

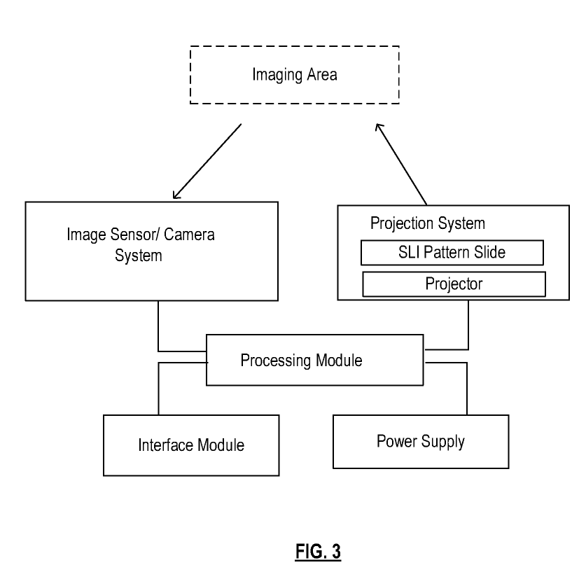

FIG. 3 illustrates a schematic block diagram of another embodiment of a Structured Light Illumination (SLI) cosmetology image sensor system. The SLI system includes an image sensor system, projection system, processing module 106, interface module and power supply. The image sensor system includes one or more image sensors operable to capture images of an anatomical feature. Projection system includes one or more projectors and one or more SLI pattern slides. Alternatively, laser lights may be programmed to project a certain SLI pattern onto the anatomical feature. The power supply is coupled to the image sensor system, projection system and processing module. The interface module provides a display and user interface, such as keyboard or mouse, for monitoring and control of the SLI system by an operator. The interface module may include other hardware devices or software needed to operate the SLI system and provide communication between the components of the SLI system.

Processing module is operable to control the cosmetology image sensor system and projection system. In general, the processing module includes one or more processing devices, such as a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module includes a memory that is an internal memory or an external memory. The memory of the processing module 106 may each be a single memory device or a plurality of memory devices. Such a memory device may be a read-only memory, random access memory, volatile memory, non-volatile memory, static memory, dynamic memory, flash memory, cache memory, and/or any device that stores digital information. When processing module may implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. Processing module may execute hard coded and/or operational instructions stored by the internal memory and/or external memory to perform the steps and/or functions illustrated in FIGS. 1 through 15 described herein. The processing module and the interface module may be integrated into one or more devices or may be separate devices.

In operation, an anatomical feature is imaged in the imaging area. The anatomical feature may move through the imaging area or the image sensor system may be moved across a body to capture the desired anatomical features.

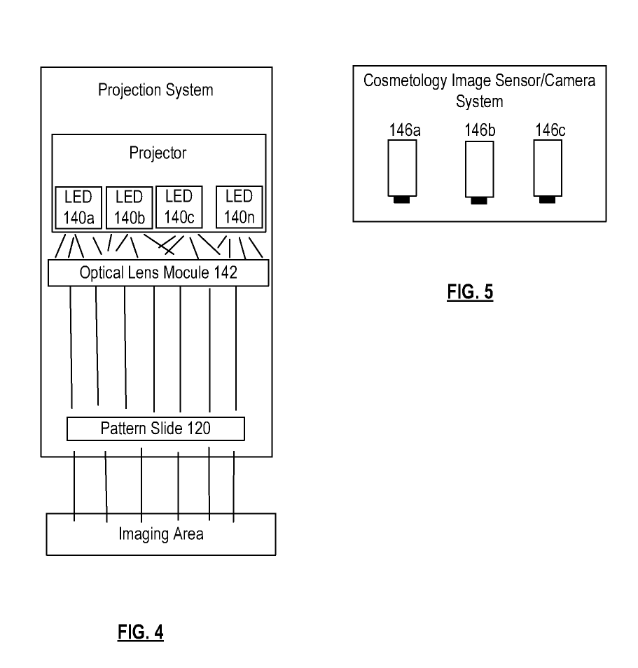

FIG. 4 illustrates a schematic block diagram of an embodiment of a projection system. In this embodiment, the projector includes an array of high intensity light emitting diodes (LED) 140a-n. The LEDs 140a-n are triggered for a pulse duration sufficient to provide ample exposure at the highest frame rate of the image sensor system 102, while minimizing the duration to avoid motion blur of the anatomical feature during the exposure. The use of an array of LEDs rather than a DLP projector in this embodiment reduces hardware cost and size of the SLI system 100. Using a high intensity LED array as a flash unit also allows for increased image signal to noise ratio (SNR) and shorter exposure times.

The projection system also includes optical lens module 142. The optical lens module 142 projects the light from the LEDs through the SLI pattern slide and focuses the SLI pattern into the imaging area. In an embodiment, the optical lens module 142 focuses light only in the axis perpendicular to the LED array, achieving further efficiency in light output by only projecting light in an aspect ratio that matches that of the pattern slide. For example, the optical lens module may be a cylindrical lens.

FIG. 5 illustrates a schematic block diagram of an embodiment of cosmetology image sensor/camera system. The image sensor system includes one or more image sensors 140. In an embodiment, the image sensors are CCD (Charge coupled device) camera modules, CMOS (Complementary metal-oxide-semiconductor) camera modules, or other type of image sensor modules. The image sensors 140 include a high speed data interface, such as USB interface, and include a trigger input for synchronization with the projection system.

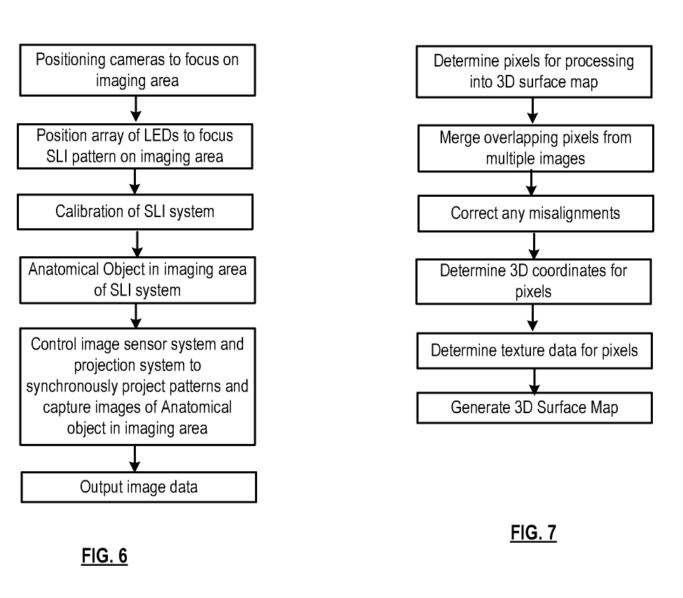

FIG. 6 illustrates a logical flow diagram of an embodiment of a method for capturing cosmetology images using SLI techniques. In operation, the image sensor/camera system is positioned and focused onto the imaging area. An SLI pattern projector projects focused light through an SLI pattern slide in imaging area. The system calibrations are taken. When an anatomical feature is positioned in the imaging area, the SLI pattern is distorted by the surface variations of the anatomical feature. While the SLI pattern is projected onto the anatomical feature, a camera captures an image of the anatomical feature with the SLI pattern distortion. The camera captures one or more images of the anatomical feature with the distortions in the structured light pattern. Additional SLI slide patterns may be projected onto the anatomical feature 110 while additional images are captured. The one or more 3D cosmetology images are then stored in a cosmetology image data file for processing.

The 3D cosmetology image processing module shown in FIG. 1 processes the 3D cosmetology image data. FIG. 7 illustrates a logical flow diagram of an embodiment of a method for generating a 3D surface map from SLI cosmetology image data. The distortions in the structured light pattern in the captured images are analyzed and calculations performed to determine a spatial measurement of various object points on the anatomical feature. This processing of the images uses well-known techniques in the industry, such as standard range-finding or triangulation methods. The triangulation angle between the camera and projected pattern causes a distortion directly related to the depth of the surface. Once these range finding techniques are used to determine the position of a plurality of points on the 3D object surface, then a 3D data representation of the 3D object can be created. An example of such calculations is described in U.S. Pat. No. 7,440,590, entitled, “System and Technique for Retrieving Depth Information about a Surface by Projecting a Composite Image of Modulated Light Patterns,” by Laurence G. Hassebrook, Daniel L. Lau, and Chun Guan filed on May 21, 2003, which is incorporated by reference here. The 3D coordinates for a plurality of object points is determined Collectively, the plurality of points result in a 3D surface map. Each point in the 3D surface map is represented by 3D coordinates, such as Cartesian (x,y,z) coordinates, spherical (r, θ, Φ) coordinates or cylindrical (y, r, θ) coordinates. In addition, each point includes texture data. Texture data includes color values, such as Red, Green and Blue values. Texture data also includes grey values or brightness values as well. The 3D cosmetology image processing module thus creates a 3D surface map of the anatomical feature based on the cosmetology image data from the SLI cosmetology image sensor. Various SLI techniques and SLI patterns may be implemented in the SLI system described herein. For example, see PCT Application No. WO2007/050776, entitled System and Method for 3D Imaging using Structured Light Illumination, which is incorporated by reference herein. See also, US Published Application No. 20090103777, entitled Lock and Hold Structured Light Illumination, which is also incorporated by reference herein. See also, PCT application Ser. No. 09/43056, entitled “System and Method for Structured Light Illumination with Frame Subwindows,” filed on May 6, 2009, which is incorporated by reference herein.

The image data is further processed by ignoring certain points while connecting other points to reduce the 3D surface map data. In an embodiment, cosmetology image processing module segments the 3D surface map to eliminate unwanted points or data. The segmentation technique includes background-foreground modeling to eliminate background image data from a region of interest. The background-foreground modeling is performed as part of a training stage by collecting a number of background images and computing the average background model image. The foreground image information is extracted by labeling any image pixel that does not lie within a specified tolerance of the average background model image. The segmented 3D shape is the 3D information for the segmented foreground image. For example, the 3D points on a surface map for a certain skin area are extracted from background points or separated from other points of the surface map.

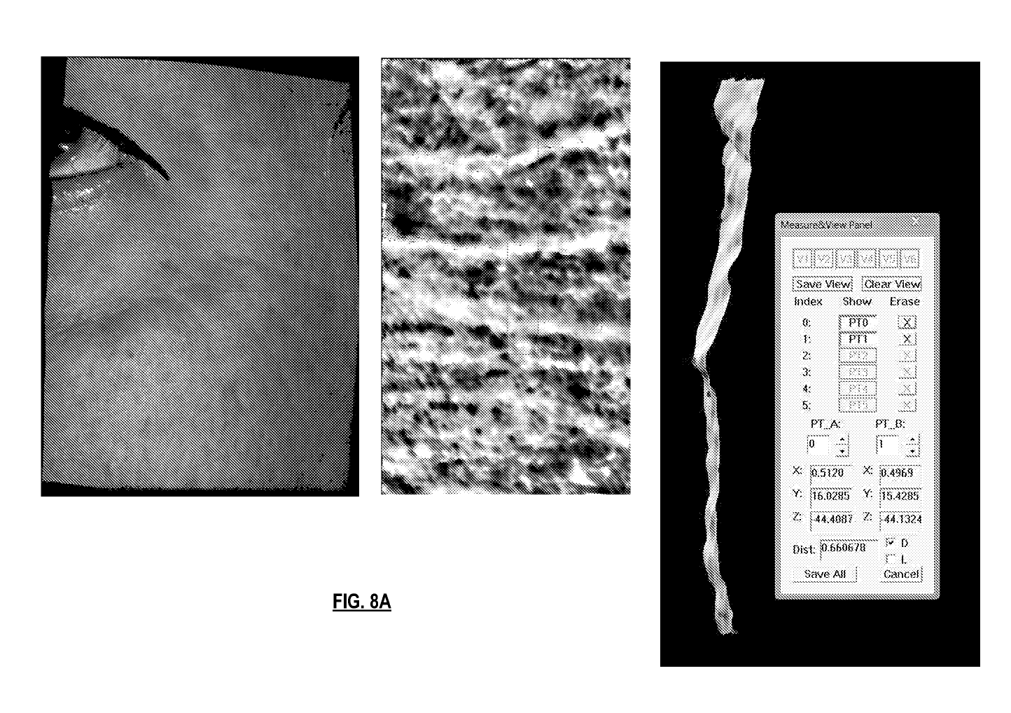

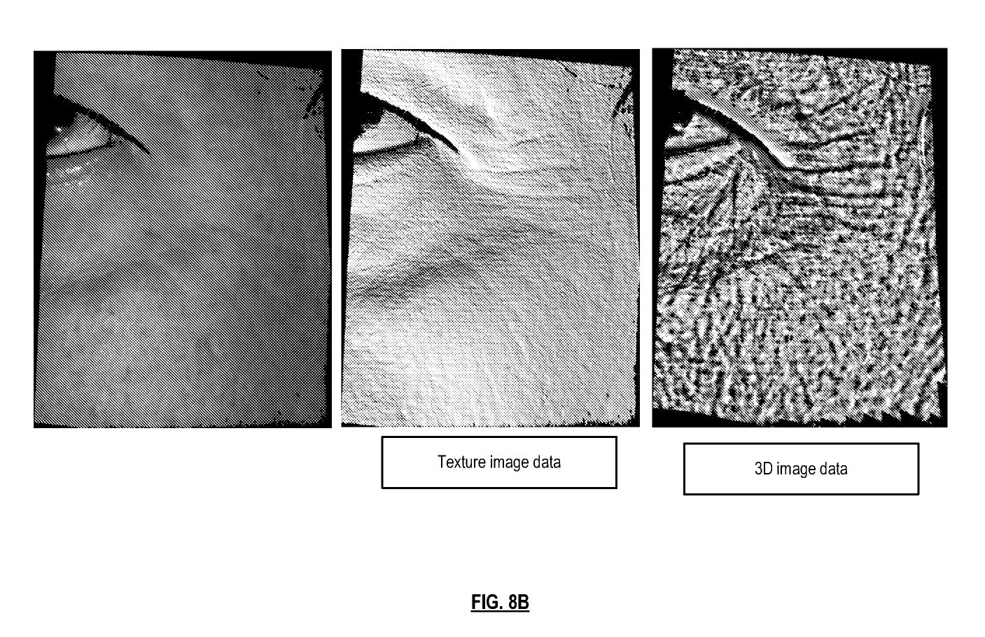

FIGS. 8A and 8B illustrate an example of 3D surface maps generated from SLI image data. In the example of FIG. 8A, the 3D surface map includes pores, wrinkles—ridges and furrows—from skin around an eye area. Since the 3D surface map includes 3D coordinates of each of the points in the surface map, the size and shape of various features can be measured, such as the size and shape of a pore or depth of a furrow. For example, FIG. 8A shows two points on the 3D surface map—a red point PT0 at a top of a ridge and a blue point PT1 near the bottom of the ridge. The figure shows the X,Y,Z coordinates for the points and the distance of 0.660678. FIG. 8B illustrates texture data, such as color and relative contrast, of a feature can also be determined from the 3D surface map.

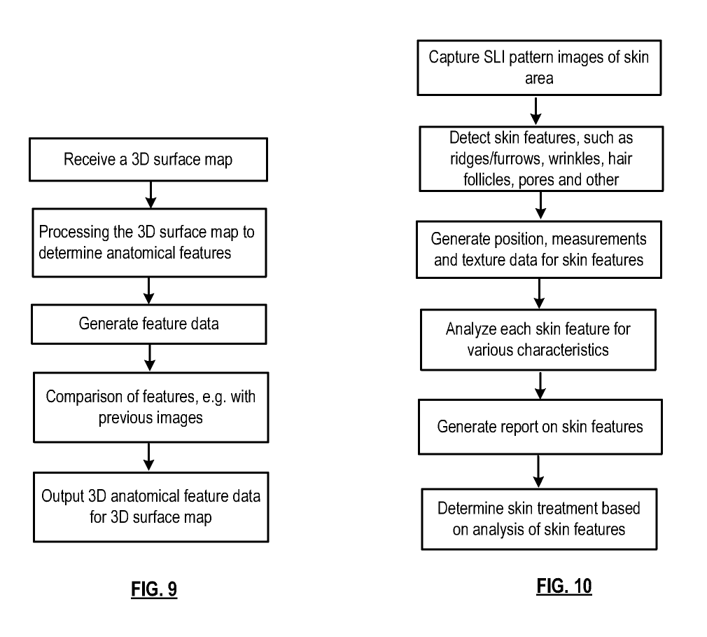

FIG. 9 illustrates a logic flow diagram of an embodiment for processing a 3D surface map to generate anatomical feature data. Once a 3D surface map is generated, characteristics of anatomical features present in the surface map can be determined and measured. Depending on type of feature, the 3D surface map is compared to various feature templates. The detected feature data is compared with feature data from previous SLI scan images to determine changes over time. Changes, such as in size, density, shape and color, can be objectively measured. The results of the comparison are provided to a cosmetology expert for interpretation and review.

In an embodiment, the anatomical feature is a skin. The feature analysis module analyzes the SLI scan images of the skin surface to detect a volume and density of wrinkles and measure changes from previous images. Color, tint, hue, contrast of skin area can also be measured. The feature analysis module may also detect damage to skin and visually demonstrates various skin conditions that need addressing. In an embodiment, the treatment analysis module may then analyze skin color, e.g. generate RGB values or other color analysis, and then match the color analysis to products for best results. For example, a skin product such as concealer or base for skin, can be matched based on the color analysis. In an embodiment, the treatment analysis may also analyze skin cells and determine hydration level. It may also analyze hair color or hair damage/condition to recommend hair treatments or proper color treatments.

FIG. 10 illustrates a logic flow diagram of an embodiment for using SLI techniques in cosmetology for skin treatments. The SLI system described herein provides a lower cost system to assist in cosmetology of skin treatments. Due to high costs, current imaging systems are not affordable for the average cosmetologist. In addition, current imaging costs are too expensive for frequent visits. Due to its lower costs, the SLI cosmetology imaging system described herein is affordable and cost effective solution for frequent imaging at a cosmetologist office or salon. In an embodiment, a cosmetic consultant may take images of customers in a store or salon to determine proper treatments and products.

The SLI cosmetology image sensor images an area of skin, and the image processing module generates a 3D surface map of the skin area. A skin feature detection module then detects skin features, such as wrinkles, color, hydration (dry, oily), freckles, and other lesions, from the 3D surface area and extracts the points for such features for further analysis. Because the 3D surface map includes 3D coordinates and texture data for each point, the SLI cosmetology imaging system can determine size measurements, density measurements, shape measurements and texture data for skin features.

A skin feature analysis module compares each skin feature for various characteristics. For example, density and depth of wrinkles can be measured in the skin area. Hydration of skin can be measured that calculates skin hydration level based on cell detail. The hydration can be compared to a chart or ranking (1-10 dryness level or type skin as dry, oily, combination, etc). Color and contrast of the skin can also be measured and specific values provided of RGB. The color and contrast may also be categorized or typed into one or more categories as well.

In an embodiment, the treatment analysis module is then used to recommend one or more skin treatments, including one or more skin products. For example, a database with a list of products and uses for such products can be accessed by the treatment analysis module and one or more products recommended. For example, various anti-aging products may be recommended based on volume, density, depth of wrinkles. Various hydration products may be recommended to increase hydration or decrease oiliness of the skin. In addition, based on a color, tint or hue analysis of the skin, various shades of cosmetics may also be recommended. In addition, the images and analysis for a skin area may be stored in a user account and compared with later images of the skin area to determine progress or effectiveness of a skin treatment.

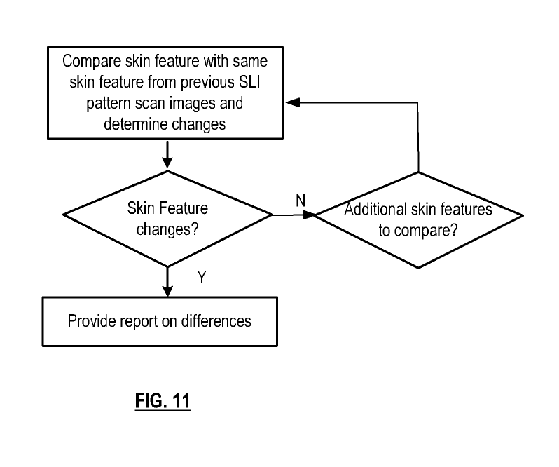

FIG. 11 illustrates a logic flow diagram of an embodiment of a method for processing skin feature data captured using SLI techniques with prior images. The SLI cosmetology imaging system detects skin features as described herein. Feature data for skin area is then compared with prior images and differences in the skin features are reported.

In an embodiment, multispectral visible light with ultraviolet light images may be taken of a skin area by the SLI imaging system for skin damage assessment. The SLI system can overlay the ultraviolet surface damage map onto the 3D surface to determine exact 3D measurements.

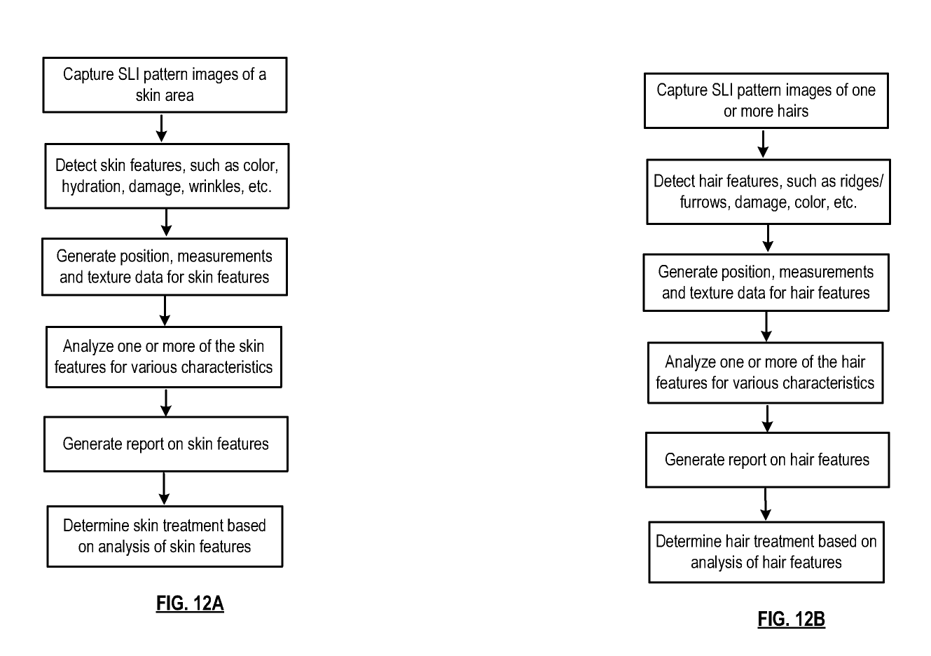

FIG. 12A illustrates a logic flow diagram of an embodiment for using SLI techniques in cosmetology for skin treatments. A 3D image is generated by the SLI scanner of an area of skin, e.g. on the face, hands, etc. Various skin features are detected and measurements determined, such as RGB values for color, density of wrinkles, etc. For example, density and depth of wrinkles can be measured in the skin area. Hydration of skin can be measured that calculates skin hydration level based on cell detail. The hydration can be compared to a chart or ranking (1-10 dryness level or type skin as dry, oily, combination, etc). Color and contrast of the skin can also be measured and specific values provided of RGB or other color values. The color and contrast may also be categorized or typed into one or more categories as well.

In an embodiment, one or more skin treatments are recommended based on the analysis, such as one or more skin products (make-up type and color, lotions, facial masks), types of facials, procedures (such as microdermabrasion, botox, etc). For example, a database with a list of products and uses for such products can be accessed by the treatment analysis module and one or more products recommended. For example, various anti-aging products may be recommended based on volume, density, depth of wrinkles. Various hydration products may be recommended to increase hydration or decrease oiliness of the skin. In addition, based on a color, tint or hue analysis of the skin, various shades of cosmetics may also be recommended. In addition, the images and analysis for a skin area may be stored in a user account and compared with later images of the skin area to determine progress or effectiveness of a skin treatment.

FIG. 12B illustrates a logic flow diagram of an embodiment for using SLI techniques in cosmetology for hair treatments. The SLI cosmetology image sensor images one or more hairs, and the image processing module generates a 3D surface map of the hair surface. A hair feature detection module then detects features of the hair, such as damage, furrows, ridges, color, hydration (dry, oily), etc. from the 3D surface area and extracts the points for such features for further analysis. Because the 3D surface map includes 3D coordinates and texture data for each point, the SLI cosmetology imaging system can determine size measurements, density measurements, shape measurements and texture data for the hair features.

A hair feature analysis module compares each feature for various characteristics. For example, damage to the hair can be determined from density and depth of furrows/ridges. Hydration of the hair can be measured that calculates hydration level. The hydration can be compared to a chart or ranking (1-10 dryness level or type skin as dry, oily, combination, etc). Color and contrast of the hair can also be measured and specific values provided, for example RGB values. The color and contrast may also be categorized or typed into one or more categories as well.

In an embodiment, the treatment analysis module is then used to recommend one or more hair treatments, including one or more hair products. For example, a database with a list of products and uses for such products can be accessed by the treatment analysis module and one or more products recommended. For example, various shampoo or conditioning products may be recommended based on damage/hydration level. In addition, based on a color, tint or hue and texture analysis of the hair, various hair dye or highlights may be recommended to obtain desired shades of hair. In addition, the images and analysis for the hair may be stored in a user account and compared with later images of the hair to determine progress or effectiveness of a treatment.

As may be used herein, the term “operable to” indicates that an item includes one or more of processing modules, data, input(s), output(s), etc., to perform one or more of the described or necessary corresponding functions and may further include inferred coupling to one or more other items to perform the described or necessary corresponding functions.

The present invention has also been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claimed invention.

The present invention has been described above with the aid of functional building blocks illustrating the performance of certain significant functions. The boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality. To the extent used, the flow diagram block boundaries and sequence could have been defined otherwise and still perform the certain significant functionality. Such alternate definitions of both functional building blocks and flow diagram blocks and sequences are thus within the scope and spirit of the claimed invention. One of average skill in the art will also recognize that the functional building blocks, and other illustrative blocks, modules and components herein, can be implemented as illustrated or by one or multiple discrete components, networks, systems, databases or processing modules executing appropriate software and the like or any combination thereof.

What is claimed is:

1. A structured light illumination (SLI) cosmetology imaging system, comprising:
an SLI image sensor that captures one or more images of an anatomical feature and generates image data of the anatomical feature;
a three dimensional (3D) image processing module that process the image data of the anatomical feature and generates a 3D surface map of the anatomical feature;
a feature detection module that processes the 3D surface map and generates feature data for predetermined characteristics of the anatomical feature;
a feature analysis module that analyzes the feature data to provide a categorization and rating of the anatomical feature based on correlations of feature characteristics; and
a treatment analysis module that processes the categorization and rating of the anatomical feature to determine a recommended cosmetology treatment.
